(19) United States
(12) Patent Application Publication
(10) Pub. No.: US 2024/0398618 A1
(43) Pub. Date: Dec. 5, 2024

(54) PHACOEMULSIFICATION SYSTEM

(71) Applicant: JOHNSON & JOHNSON SURGICAL VISION, INC., Irvine, CA (US)

(72) Inventors: Vadim Gliner, Haifa (IL); Eran Haas, Haifa (IL); Amit Fuchs, Hogla (IL)

(21) Appl. No.: 18/203,673

(22) Filed: May 31, 2023

(51) Int. Cl. A61F 9/007; A61M 1/00

(52) U.S. Cl. CPC A61F 9/00745; A61M 1/74

(57) ABSTRACT

Phacoemulsification apparatus, consisting of a phacoemulsification probe having a distal end including a combination of a needle and a sleeve, the combination being configured for insertion into an eye of a patient, and being configured for fluid transfer between the eye and the combination. The apparatus has an ultrasound actuator configured to vibrate the needle, a first sensor configured to output a first indication indicative of physical contact between the needle and a lens of the eye, and a second sensor configured to output a second indication indicative of a parameter associated with the fluid transferred by the combination. A microcontroller is configured to receive the first and the second indication, to calculate a threshold for the first indication in response to the second indication, and to activate and deactivate the ultrasound actuator in response to the first indication crossing the threshold.

### FIELD OF THE DISCLOSURE

This disclosure relates generally to ocular surgery, and specifically to the efficacy of a phacoemulsification system used for the surgery.

### BACKGROUND

A cataract is a cloudy area in the lens of the eye that leads to a decrease in vision. Phacoemulsification is a modern cataract surgery method in which the eye's internal lens is emulsified with an ultrasonic handpiece and aspirated from the eye. In some cases, the ultrasonic energy used may be responsible for edema in the eye.

### BRIEF DESCRIPTION OF THE DRAWINGS

The present disclosure will be understood from the following detailed description, taken in conjunction with the drawings in which:

FIG. 1 is a pictorial view of a phacoemulsification apparatus, according to an example of the present disclosure;

FIG. 2 is a block diagram of elements of a phacoemulsification probe/handpiece that is a part of the apparatus of FIG. 1, according to an example of the present disclosure;

FIG. 3 is a flowchart of steps implemented to modulate power delivered to an actuator during a phacoemulsification procedure, according to an example of the present disclosure; and

FIG. 4 is a power vs. time graph illustrating the power modulation of FIG. 3, according to an example of the present disclosure.

### DESCRIPTION OF EXAMPLES

### Overview

In a phacoemulsification procedure, assumed herein to be performed to remove the lens of a patient's eye, a surgeon uses a phacoemulsification handpiece to insert a needle into the eye. The needle is hollow and has a small diameter lumen. The needle is navigated so that it contacts the eye lens, and it is vibrated ultrasonically by a piezoelectric actuator in the handpiece, causing the lens to break into particles. An aspiration pump aspirates the particles, in aspiration fluid withdrawn from the eye via an aspiration line coupled with the needle lumen. To maintain intraocular pressure during the procedure, an irrigation pump irrigates the eye with irrigation fluid via an irrigation line and a sleeve surrounding the needle; the sleeve and the needle it surrounds are herein also termed a needle-sleeve combination. The aspiration and irrigation pumps may be housed in or outside a console used for the procedure.

The ultrasonic vibration of the needle dissipates energy in the eye, and when the needle contacts the lens, this energy breaks the lens into particles, as is described above. However, if the needle is not in contact with the lens, any ultrasonic vibration continues to dissipate energy. Apart from wasting energy when the needle is not in contact with the lens, the energy from the ultrasonic vibrations may have unwanted side effects, such as overheating of ocular tissue, and/or causing macular or corneal edema. It is thus more efficient, as well as being safer, if the needle is only vibrated when it is in contact with the lens.

When the needle contacts the lens, the pressure of the aspiration fluid, measured at a distal tip of the needle-sleeve combination, reduces, compared to the tip pressure of the aspiration fluid when there is no lens contact. Similarly, when the needle contacts the lens, the pressure of the irrigation fluid at the distal tip increases, compared to the irrigation fluid tip pressure when there is no contact.

Examples of the present disclosure detect when the needle contacts the lens, and reduce or completely halt the ultrasonic vibrations if the needle does not contact the lens. In examples of the present disclosure the contact is determined if a measured parameter associated with the irrigation tip pressure or the aspiration tip pressure crosses a dynamic pressure threshold. The dynamic pressure threshold is a varying quantity formed, inter alia, from a registered value of a property, such as a flow rate, a vacuum level, or a pressure level, of one of the fluids.

In a disclosed example the measured parameter is the aspiration fluid pressure measured in the handpiece, i.e., at a proximal location distant from the distal tip. The dynamic pressure threshold is a function of the flow rate of the aspiration fluid, and is also a function of the diameter of the needle lumen.

The handpiece aspiration fluid pressure is measured by an aspiration sensor in the aspiration line of the handpiece. The sensor provides a signal, indicative of the handpiece aspiration fluid pressure, to a microcontroller which may be integrated into the handpiece. Alternatively, the microcontroller may be integrated into a disposable unit (e.g., an anti-vacuum structure) coupled with the handpiece, or be located in the surgical console.

The microcontroller is provided with a preset function relating the dynamic pressure threshold to the aspiration fluid flow rate and the needle lumen diameter. The microcontroller uses the function to calculate a value of the dynamic pressure threshold, and activates or deactivates the piezoelectric actuator depending on whether or not the handpiece aspiration fluid pressure exceeds the dynamic pressure threshold.

The aspiration line sensor is necessarily distant from the distal tip of the needle-sleeve combination, because of the small dimensions of the needle and its associated sleeve. In examples of the present disclosure, the change of threshold provided by the dynamic pressure threshold compensates for the distant positioning of the aspiration line sensor.

In an alternative disclosed example, the measured parameter is the irrigation fluid pressure measured by an irrigation sensor in the handpiece, and the dynamic pressure threshold is a function of the flow rate of the irrigation fluid.

### System Description

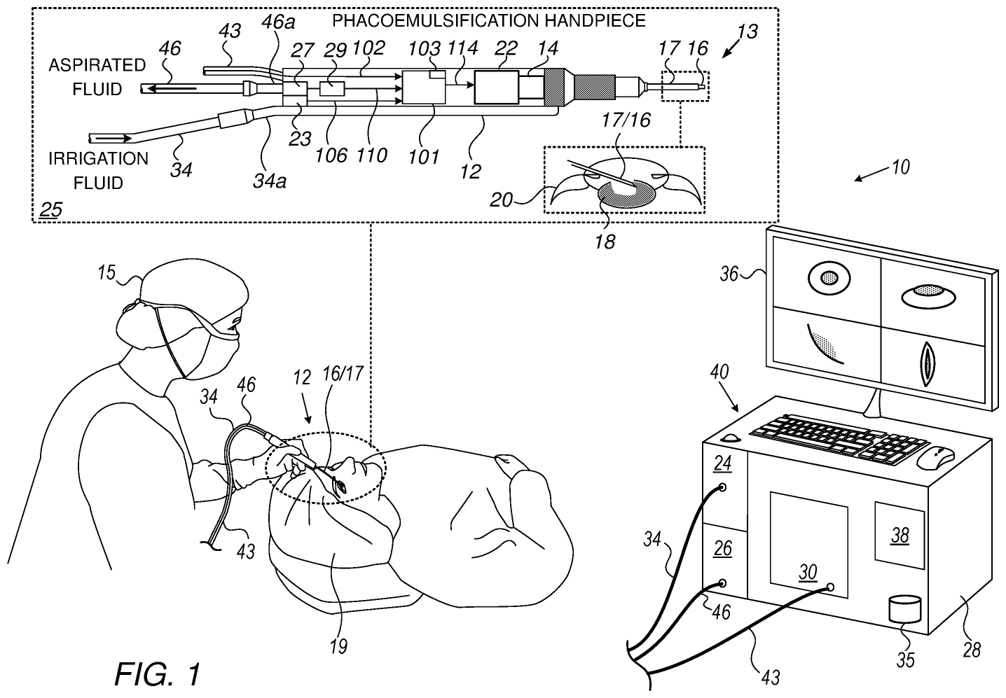

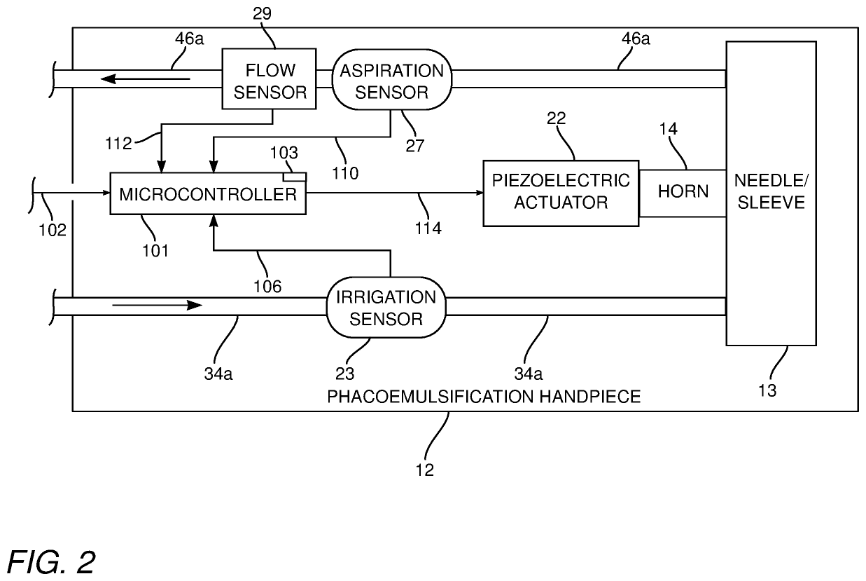

FIG. 1 is a pictorial view of a phacoemulsification apparatus 10, and FIG. 2 is a block diagram of elements of a phacoemulsification probe/handpiece 12 that is a part of the apparatus, according to an example of the present disclosure.

FIG. 1 includes an inset 25, and as shown in the figure and the inset apparatus 10 includes phacoemulsification probe/handpiece 12 comprising a hollow needle 16 and a coaxial irrigation sleeve 17. Irrigation sleeve 17 at least partially surrounds the needle 16 and creates a fluid pathway between the external wall of the needle 16 and the internal wall of the sleeve 17. Needle 16 and sleeve 17 are also herein termed a needle-sleeve combination 13.

Needle 16 is configured to be inserted into a lens 18 of an eye 20 of a patient 19. Needle 16 is mounted on a horn 14 of probe 12, and is shown in inset 25 as a straight needle. However, any suitable needle may be used with the phacoemulsification probe 12, for example, a curved or bent tip needle that is commercially available from Johnson & Johnson Surgical Vision, Inc., Irvine, CA, USA.

A physician 15 holds handpiece 12 so as to perform a phacoemulsification procedure on the eye 20 of patient 19. The physician may activate the handpiece using a foot pedal (not shown in the figures). Handpiece 12 comprises a piezoelectric actuator 22, which is configured to vibrate horn 14 and needle 16 in one or more vibration modes of the combined horn and needle. During the phacoemulsification procedure the vibration of needle 16 is used to break up a natural lens 18 into small pieces.

Elements of apparatus 10 are under overall control of a processor 38 in a console 28. Functions of processor 38 are described in more detail below.

During the phacoemulsification procedure, an irrigation pump 24, which may be in or outside console 28, pumps irrigation fluid through an irrigation channel 34a in handpiece 12 to irrigation sleeve 17 so as to irrigate the eye. The fluid is pumped via an irrigation tubing line 34, running from the pump, that is connected to channel 34a of the probe 12. An aspiration pump 26, which also may be located in or outside console 28, aspirates aspiration fluid, comprising eye fluid and waste matter (e.g., emulsified parts of the lens), from the patient's eye 20 via needle 16, through an aspiration channel 46a in handpiece 12. Aspiration pump 26 produces a vacuum that is connected from the pump to aspiration channel 46a by a vacuum aspiration tubing line 46.

Pumps 24 and 26 may be any pump known in the art (e.g., a peristaltic pump or a progressive cavity pump), and the pumps are both under overall control of processor 38. Some or all of the functions of processor 38 may be combined in a single physical component or, alternatively, implemented using multiple physical components. The physical components may comprise hard-wired or programmable devices, or a combination of the two.

An aspiration sensor 27 in a proximal section of probe 12 is coupled with the aspiration fluid, and the sensor provides signals to processor 38 enabling the processor to control the pump rate of aspiration pump 26. In an example, aspiration sensor 27 may be connected to aspiration channel 46a in handpiece 12, as is illustrated in the figures. Alternatively, aspiration sensor 27 may be coupled with aspiration tubing line 46.

An irrigation sensor 23 in the proximal section of probe 12 is coupled with the irrigation fluid, and the sensor provides signals to processor 38 enabling the processor to control the pump rate of irrigation pump 24. As is illustrated in the figures, irrigation sensor 23 may be connected to irrigation channel 34a in handpiece 12. Alternatively, irrigation sensor 23 may be coupled with irrigation tubing line 34.

Irrigation sensor 23 and aspiration sensor 27 may be any sensor known in the art that may be configured to be a vacuum sensor or a pressure sensor.

In an example a flow sensor 29 in a proximal section of probe 12 may be coupled with the aspiration fluid, and the sensor may provide signals indicative of a flow rate to processor 38. Alternatively, processor 38 may determine the flow rate from its control of aspiration pump 26, e.g., using an encoder to count the pump revolutions to determine the flow rate, in which case apparatus 10 may not have flow sensor 29. Flow sensor 29 may use the Doppler effect to measure a flow rate. In an example, flow sensor 29 may be connected to aspiration channel 46a in handpiece 12, as is illustrated in the figures. Alternatively, flow sensor 29 may be coupled with aspiration tubing line 46.

Actuator 22 is powered by a driving module 30 in console 28. Module 30, under overall control of processor 38, is configured to provide the power to the actuator, via a microcontroller 101, as a resonant radio-frequency (RF) driving current. Microcontroller 101 has an associated memory 103, which the microcontroller may use in performing its functions. In the illustrated example the RF current from module 30 is delivered via cable 43 to microcontroller 101 in handpiece 12. In alternative examples microcontroller 101 may be integrated into a disposable unit coupled with the handpiece (e.g., an anti-vacuum surge unit), or the microcontroller may be located in console 28.

Microcontroller 101 is configured to modulate the driving current it delivers to actuator 22, so that the power dissipated by the actuator is reduced when needle 16 does not contact lens 18. The method of modulation is described below, the microcontroller is selected and configured so that the modulation may have a fast reaction time, of the order of 0.1 microseconds. When microcontroller 101 is located in the handpiece, or in a unit coupled with the handpiece, it facilitates fast reaction times.

In addition to the signals from irrigation sensor 23, aspiration sensor 27, and flow sensor 29 being provided to processor 38, so that the processor can control irrigation pump 24 and aspiration pump 26, the signals are also provided to microcontroller 101. In FIGS. 1 and 2, an arrow 102 illustrates the RF driving power delivered from module 30 to microcontroller 101, arrows 106, 110, and 112 respectively illustrate the signals from irrigation sensor 23, aspiration sensor 27, and flow sensor 29 delivered to the microcontroller, and an arrow 114 illustrates the modulated RF driving power delivered from the microcontroller to actuator 22.

At least some of the functions of processor 38 may be carried out by suitable software stored in a memory 35. The software may be downloaded to a device in electronic form, over a network, for example. Alternatively, or additionally, the software may be stored in tangible, non-transitory computer-readable storage media, such as optical, magnetic, or electronic memory.

Processor 38 may receive user-based commands via a system user interface 40, which may include setting and/or adjusting a vibration mode and/or a frequency of piezoelectric actuator 22, setting and/or adjusting a stroke amplitude of needle 16, and setting and/or adjusting a default irrigation rate and a default aspiration rate of irrigation pump 24 and aspiration pump 26.

Additionally, or alternatively, processor 38 may receive user-based commands from controls located in handpiece 12, to, for example, select a trajectory for needle 16.

Processor 38 may present setting and parameter information of the phacoemulsification procedure on a display 36. In an example, user interface 40 and display 36 may be one and the same, such as a touch screen graphical user interface.

The procedure illustrated in FIG. 1 may include further elements, which are omitted for clarity of presentation. For example, physician 15 typically performs the procedure using a stereo-microscope or magnifying glasses, neither of which are shown. Physician 15 may use other surgical tools, in addition to probe 12, which are also not shown to maintain clarity and simplicity.

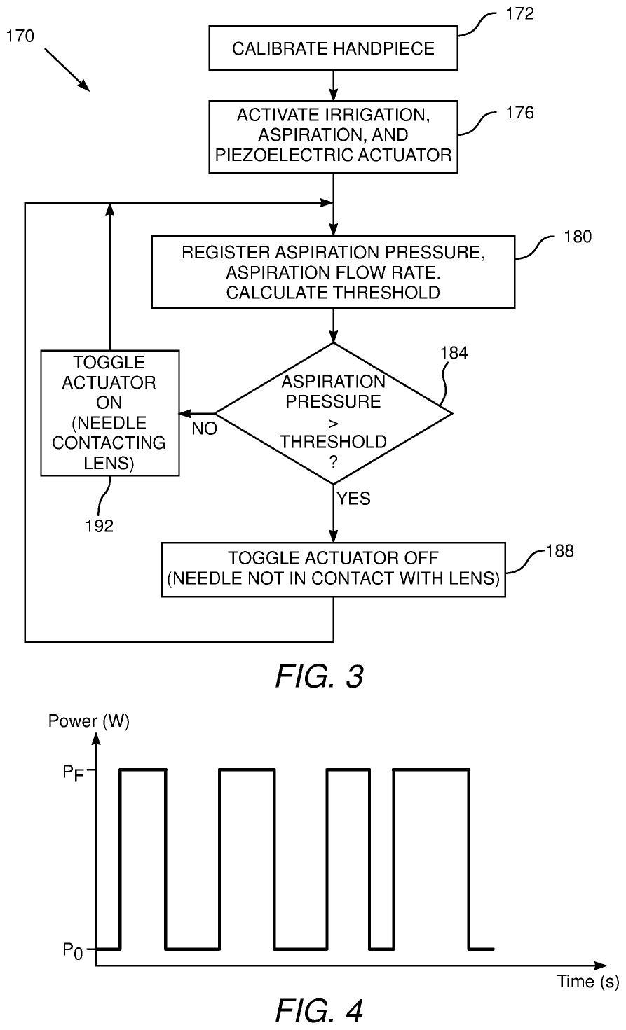

FIG. 3 is a flowchart 170 of steps implemented to modulate the power delivered to actuator 22 during the phacoemulsification procedure referred to above, and FIG. 4 is a power vs. time graph illustrating the modulation, according to an example of the present disclosure. As is illustrated in the graph, the modulation provided by microcontroller 101 varies the delivered power from an approximately full power level P_{F} to a reduced power level P₀; the reduced power level may comprise zero power. Microcontroller 101 is configured to apply full power level P_{F} when needle 16 is in contact with lens 18, and to apply reduced power level P₀ when the needle is not in contact with the lens.

In examples of the present disclosure, microcontroller 101 determines if there is contact or no contact with lens 18 by finding if a parameter derived from characteristics of either the aspiration fluid or the irrigation fluid is above or below a dynamic pressure threshold value. The dynamic pressure threshold value is also derived, at least in part, from characteristics of either of the fluids.

For simplicity and clarity, except where otherwise stated, the following description is directed to examples using parameters derived from the aspiration fluid. Those having ordinary skill in the art will be able to adapt the description, mutatis mutandis for examples using parameters derived from the irrigation fluid. In the description, it will be understood that pressure values, for both the aspiration fluid and the irrigation fluid, are assumed to be positive.

In a disclosed example, the parameter referred to above comprises a handpiece aspiration fluid pressure, and microcontroller 101 measures the handpiece pressure, i.e., the aspiration fluid pressure at the location of the sensor, using signals from aspiration sensor 27. It will be understood that, because of the location of sensor 27, the handpiece pressure measured by the sensor is different from the pressure of the aspiration fluid at a distal tip of needle 16, i.e., at the location where the needle contacts lens 18.

The dynamic pressure threshold value that microcontroller 101 compares with the handpiece pressure compensates for the difference of the handpiece pressure from the distal tip pressure. Thus, rather than having a fixed value for the threshold, the dynamic pressure threshold value is calculated according to the flow rate of the aspiration fluid, and the diameter of the lumen of needle 16. Incorporating the values of the flow rate and the needle dimensions into the threshold value compensates for the different locations of the needle distal tip and the aspiration sensor 27.

In a disclosed example, microcontroller 101 calculates the dynamic pressure threshold value according to equation (1):

$$T = {F}_{1} ( G , {I}_{A S P} ) \qquad ( 1 )$$

where T is the dynamic pressure threshold value,
G is the gauge, i.e., the internal diameter, of needle 16,
I_{ASP} is the flowrate of the aspiration fluid, and
F₁ is a function, which may or may not be analytic, of the gauge G and the flowrate I.

Equation (1) illustrates that the dynamic pressure threshold value, T, is dependent on the values of the gauge G of needle 16, and the flowrate I_{ASP} of the aspiration fluid.

In another disclosed example, the function F₁ of equation (1) is assumed to be as is given in equation (2):

$$T = G \cdot {F}_{2} ( {I}_{A S P} ) \qquad ( 2 )$$

where F₂ is a polynomial of flowrate I_{ASP}, i.e.,

$${F}_{2} ( {I}_{A S P} ) ≡ {\sum }_{k = 0}^{k = n} {a}_{k} \cdot {I}_{A S P}^{k} \qquad ( 3 )$$

where aₖ is the coefficient of I_{ASP}^{k}.

Equation (2) illustrates that in this example the dynamic pressure threshold value T is linearly dependent on gauge G, and is also linearly dependent on polynomial F₂(I_{ASP}).

In one example polynomial F₂(I_{ASP}) is of degree three, i.e.,

$${F}_{2} ( {I}_{A S P} ) ≡ {a}_{3} \cdot {I}_{A S P}^{3} + {a}_{2} \cdot {I}_{A S P}^{2} + {a}_{1} \cdot {I}_{A S P} + {a}_{0} \qquad ( 4 )$$

where a₃, a₂, a₁, a₀ are numerical coefficients of the polynomial.

In other examples the degree of polynomial F₂(I_{ASP}) is different from three, i.e., is larger or smaller than three.

Turning now to flowchart 170, in a calibration step 172 the parameters to be used by microcontroller 101 in determining the dynamic pressure threshold T are stored in a memory. The memory may be memory 103 associated with microcontroller 101, or alternatively or additionally another memory such as memory 35.

The parameters stored depend on how T is to be calculated. For example, if T is to be calculated using equation (2), then the parameters stored are values of G, the internal diameter of needle 16, for any of the needles which may be attached to handpiece 12. The parameters stored also include the coefficients of the terms of the polynomial F₂(I_{ASP}).

If T is to be calculated using equation (1), then the parameters stored depend on the type of function F₁ defining T, and those having ordinary skill in the art will be able to determine the required parameters according to the function type.

In some examples, for example if F₁ is not an analytic function, the parameters for determining T may be stored as a look-up table of values of G and I_{ASP}. If a look-up table is used, then microcontroller 101 may apply interpolation and/or extrapolation to determine the value of T if the value is not directly calculable from the look-up table.

In calibration step 172 the parameters for determining T may be stored during a handpiece priming phase of the phacoemulsification procedure. Alternatively or additionally, the parameters for determining T may be stored when probe 12 is produced.

The calibration is performed by simulating needle 16 to be in contact with lens 18, for example by inserting the distal tip of the needle into a porous material. During the simulation processor 38 operates aspiration pump 26 to have different values of I_{ASP}, and for each value of I_{ASP} the signal from aspiration sensor 27 is recorded. In some examples, a lookup table relating I_{ASP} to an externally measured flow rate is first generated, and the calibration is performed using the externally measured flow rate.

The recorded signal values and the values of I_{ASP}, as determined directly or from the lookup table referred to above, as well as the value of G, are used to evaluate coefficients of function F₁ or F₂ if the function is an analytic function, and the coefficients are then stored. Alternatively, for example if F₁ is not an analytic function, the values of I_{ASP}, G, and the signal values are stored in a look-up table. In the following steps of flowchart 170, microcontroller 101 uses the stored values to calculate dynamic pressure threshold T.

In an activation step 176, physician 15 activates, as desired, irrigation pump 24, aspiration pump 26, and actuator 22.

In a measurement step 180, which is an initial step of an iterative process comprising the following steps, microcontroller 101 registers the signal received from aspiration sensor 27, and calculates the handpiece aspiration pressure from the signal.

In an example processor 38 may act as a flow rate sensor by providing microcontroller 101 with the aspiration flow rate generated by aspiration pump 26. Alternatively, microcontroller 101 may use signals from flow sensor 29, via line 112, to determine the flow rate. The microcontroller uses the flow rate and the parameters stored in calibration step 172 to calculate the dynamic pressure threshold value T.

In a comparison step 184 microcontroller 101 checks if the handpiece aspiration pressure is greater than the dynamic pressure threshold value T.

If the comparison in step 184 returns positive, i.e., the aspiration pressure is greater than the threshold value, so that needle 16 is not in contact with lens 18, the microcontroller toggles the actuator off, in a first toggle step 188. Toggling the actuator off causes the power dissipated by the actuator to be P₀, as illustrated in FIG. 4.

If the comparison in step 184 returns negative, i.e., the aspiration pressure is less than or equal to the threshold value, so that needle 16 is in contact with lens 18, the microcontroller toggles the actuator on, in a second toggle step 192. Toggling the actuator on causes the power dissipated by the actuator to be P_{F}.

From steps 188 and 192 the flowchart returns to measurement step 180, so that steps 180-192 reiterate while the phacoemulsification procedure is performed.

The description above has been directed for examples using parameters derived from the aspiration fluid. Those having ordinary skill in the art will be able to adapt the description for examples that use parameters derived from the irrigation fluid, and all such adaptations are assumed to be comprised in the scope of the present disclosure.

Thus, in equations (1)-(4), the parameter I_{ASP} may be replaced by I_{IRR}, where I_{IRR} represents the flow rate from irrigation pump 24, and the parameter G (the internal diameter of needle 16) may be replaced by the difference of the inner diameter of sleeve 18 and the outer diameter of needle 16. Furthermore, irrigation sensor 23 replaces aspiration sensor 27.

Also, in examples using irrigation fluid parameters, the comparison of step 184 is altered to accommodate the fact that the irrigation fluid pressure is greater than a threshold value when needle 16 is in contact with lens 18. This is in contrast to the aspiration case, where the aspiration fluid pressure is less than or equal to a threshold value when there is needle-lens contact.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

### EXAMPLES

Example 1. A phacoemulsification apparatus, comprising: a phacoemulsification probe (12) having a distal end comprising a combination (13) of a needle (16) and a sleeve (17), the combination being configured for insertion into an eye (20) of a patient (19), and being configured for fluid transfer between the eye and the combination; an ultrasound actuator (22) configured to vibrate the needle; a first sensor (27) configured to output a first indication indicative of physical contact between the needle and a lens of the eye; a second sensor (29) configured to output a second indication indicative of a parameter associated with the fluid transferred by the combination; and a microcontroller (101), which is configured to receive the first and the second indication, to calculate a threshold for the first indication in response to the second indication, and to activate and deactivate the ultrasound actuator in response to the first indication crossing the threshold.

Example 2. The apparatus according to example 1, wherein the fluid transferred comprises aspiration fluid transferred from the eye to the combination.

Example 3. The apparatus according to any one of examples 1 to 2, wherein the first sensor comprises a pressure sensor, located in a proximal section of the phacoemulsification probe, and wherein the first indication comprises a signal indicative of an aspiration fluid pressure.

Example 4. The apparatus according to any one of examples 1 to 3, wherein the parameter comprises an aspiration fluid flow rate.

Example 5. The apparatus according to any one of examples 1 to 4, wherein the threshold comprises a function of the aspiration fluid flow rate and an internal diameter of the needle.

Example 6. The apparatus according to any one of examples 1 to 5, wherein the second sensor comprises a flow rate sensor.

Example 7. The apparatus according to any one of examples 1 to 6, wherein the fluid transferred comprises irrigation fluid transferred to the eye from the combination.

Example 8. The apparatus according to any one of examples 1 to 7, wherein the first sensor comprises a pressure sensor, located in a proximal section of the phacoemulsification probe, and wherein the first indication comprises a signal indicative of an irrigation fluid pressure.

Example 9. The apparatus according to any one of examples 1 to 8, wherein the parameter comprises an irrigation fluid flow rate.

Example 10. The apparatus according to any one of examples 1 to 9, wherein the threshold comprises a function of the irrigation fluid flow rate and a difference in an inner diameter of the sleeve and an outer diameter of the needle.

Example 11. A method for controlling a phacoemulsification system, comprising: providing a phacoemulsification system having a probe (12) having a distal end comprising a combination (13) of a needle (16) and a sleeve (17), an ultrasound actuator (22), a first sensor (27), and a second sensor (29), wherein the combination is configured for insertion into an eye (20) of a patient (19), and configured for fluid transfer between the eye and the combination, wherein the ultrasound actuator is configured to vibrate the needle, wherein the first sensor is configured to output a first indication indicative of physical contact between the needle and a lens of the eye, and wherein the second sensor is configured to output a second indication indicative of a parameter associated with the fluid transferred by the combination; transferring fluid by the combination; receiving the first and the second indication; calculating a threshold for the first indication in response to the second indication; and activating and deactivating the ultrasound actuator in response to the first indication crossing the threshold.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. Phacoemulsification apparatus, comprising:
a phacoemulsification probe having a distal end comprising a combination of a needle and a sleeve, the combination being configured for insertion into an eye of a patient, and being configured for fluid transfer between the eye and the combination;
an ultrasound actuator configured to vibrate the needle;
a first sensor configured to output a first indication indicative of physical contact between the needle and a lens of the eye;
a second sensor configured to output a second indication indicative of a parameter associated with the fluid transferred by the combination; and
a microcontroller, which is configured to receive the first and the second indication, to calculate a threshold for the first indication in response to the second indication, and to activate and deactivate the ultrasound actuator in response to the first indication crossing the threshold.

2. The apparatus according to claim 1, wherein the fluid transferred comprises aspiration fluid transferred from the eye to the combination.

3. The apparatus according to claim 2, wherein the first sensor comprises a pressure sensor, located in a proximal section of the phacoemulsification probe, and wherein the first indication comprises a signal indicative of an aspiration fluid pressure.

4. The apparatus according to claim 2, wherein the parameter comprises an aspiration fluid flow rate.

5. The apparatus according to claim 4, wherein the threshold comprises a function of the aspiration fluid flow rate and an internal diameter of the needle.

6. The apparatus according to claim 4, wherein the second sensor comprises a flow rate sensor.

7. The apparatus according to claim 1, wherein the fluid transferred comprises irrigation fluid transferred to the eye from the combination.

8. The apparatus according to claim 7, wherein the first sensor comprises a pressure sensor, located in a proximal section of the phacoemulsification probe, and wherein the first indication comprises a signal indicative of an irrigation fluid pressure.

9. The apparatus according to claim 7, wherein the parameter comprises an irrigation fluid flow rate.

10. The apparatus according to claim 9, wherein the threshold comprises a function of the irrigation fluid flow rate and a difference in an inner diameter of the sleeve and an outer diameter of the needle.

11. A method for controlling a phacoemulsification system, comprising:
providing a phacoemulsification system having a probe having a distal end comprising a combination of a needle and a sleeve, an ultrasound actuator, a first sensor, and a second sensor,
wherein the combination is configured for insertion into an eye of a patient, and configured for fluid transfer between the eye and the combination,
wherein the ultrasound actuator is configured to vibrate the needle,
wherein the first sensor is configured to output a first indication indicative of physical contact between the needle and a lens of the eye, and
wherein the second sensor is configured to output a second indication indicative of a parameter associated with the fluid transferred by the combination;
transferring fluid by the combination;
receiving the first and the second indication;
calculating a threshold for the first indication in response to the second indication; and
activating and deactivating the ultrasound actuator in response to the first indication crossing the threshold.

12. The method according to claim 11, wherein the fluid transferred comprises aspiration fluid transferred from the eye to the combination.

13. The method according to claim 12, wherein the first sensor comprises a pressure sensor, located in a proximal section of the phacoemulsification probe, and wherein the first indication comprises a signal indicative of an aspiration fluid pressure.

14. The method according to claim 12, wherein the parameter comprises an aspiration fluid flow rate.

15. The method according to claim 14, wherein the threshold comprises a function of the aspiration fluid flow rate and an internal diameter of the needle.

16. The method according to claim 14, wherein the second sensor comprises a flow rate sensor.

17. The method according to claim 11, wherein the fluid transferred comprises irrigation fluid transferred to the eye from the combination.

18. The method according to claim 17, wherein the first sensor comprises a pressure sensor, located in a proximal section of the phacoemulsification probe, and wherein the first indication comprises a signal indicative of an irrigation fluid pressure.

19. The method according to claim 18, wherein the parameter comprises an irrigation fluid flow rate.

20. The method according to claim 19, wherein the threshold comprises a function of the irrigation fluid flow rate and a difference in the inner diameter of the sleeve and the outer diameter of the needle.
